SEQUENCE LISTING (3 sequences)

SEQ ID NO: 1 - protein, 5 aa, Artificial Sequence
  Synthetic peptide linker
GGGGS

SEQ ID NO: 2 - protein, 10 aa, Artificial Sequence
  Synthetic peptide linker
GGGGSGGGGS

SEQ ID NO: 3 - protein, 15 aa, Artificial Sequence
  Synthetic peptide linker
GGGGSGGGGSGGGGS

(19) United States
(12) Patent Application Publication
(10) Pub. No.: US 2018/0028630 A1
(43) Pub. Date: Feb. 1, 2018

(54) COMPOSITIONS AND METHODS FOR THERAPEUTIC ANTI-CANCER VACCINATION

(71) Applicant: Lan Bo Chen, Lexington, MA (US)

(72) Inventor: Lan Bo Chen, Lexington, MA (US)

(21) Appl. No.: 15/664,065

(22) Filed: Jul. 31, 2017

Related U.S. Application Data
(60) Provisional application No. 62/368,567, filed on Jul. 29, 2016.

(51) Int. Cl. A61K 39/00; A61K 39/39; C07K 14/705

(52) U.S. Cl. CPC A61K 39/0011; C07K 14/705; A61K 39/39; A61K 2039/6037; A61K 2039/575; A61K 2039/572; A61K 47/646

(57) ABSTRACT

An immunogenic composition that includes a glycoconjugate containing a fusion protein composed of an immunoglobulin gamma Fc domain fused to a tumor-associated antigen, the fusion protein being cross-linked to an azido-modified stage-specific embryonic antigen 4 conjugated to diphtheria toxoid cross-reactive material 197; and α-galactosylceramide C34 or α-glucosylceramide C34. Also provided are methods for treating cancer by administering the immunogenic composition to a patient.

### CROSS-REFERENCE TO RELATED APPLICATIONS

The present application claims priority to Provisional Application No. 62/368,567, filed on Jul. 29, 2016. The content of this prior application is hereby incorporated by reference in its entirety.

### BACKGROUND

Many types of tumor cells express antigens that are not found in normal cells. These antigens, known as tumor-associated antigens (“TAAs”), have been intensively studied as targets for therapeutic anti-cancer vaccines.

For example, peptide epitopes derived from TAAs are loaded into a patient's dendritic cells ex vivo and the cells re-infused into the patient in attempts to stimulate an immune response against the tumor. In another example, whole tumor lysates are used as a source of TAAs for vaccination.

Vaccines based on protein TAAs have several drawbacks that limit their effectiveness, among them being low immunogenicity of the TAAs, selective survival of tumor cells lacking the TAAs, and loss of epitopes on the TAAs via somatic mutation.

Aside from protein TAAs, tumor cells also overexpress carbohydrate antigens on their surface. These carbohydrate TAAs, which are involved in both adhesion and metastasis of cancer cells, have also been studied as anti-cancer vaccine candidates. However, carbohydrate antigens alone typically do not stimulate a robust immune response.

The effectiveness as an anti-cancer vaccine of several carbohydrate antigens, e.g., Globo H, stage-specific embryonic antigen 3 (“SSEA3”), and stage-specific embryonic antigen 4 (“SSEA4”), has been improved by (i) fusion of the carbohydrate to a carrier protein, (ii) chemical modification of carbohydrate residues, and (iii) co-administration of the carbohydrate antigens with glycolipid adjuvants. See Lee et al. 2014, J. Am. Chem. Soc. 136:16844-16853 (“Lee et al.”). Glycolipid adjuvants are thought to boost the immune response, in part, via activation of invariant NKT cells.

There is a need for improved anti-cancer vaccines that promote more effective killing of cancer cells and provide long-lasting resistance to cancer relapse.

### SUMMARY

To meet this need, an immunogenic composition for treating cancer is provided. The immunogenic composition includes a glycoconjugate containing (1) a fusion protein composed of an immunoglobulin gamma Fc domain (“Fcγ”) fused to a tumor-associated antigen (“TAA”), the fusion protein being cross-linked to an azido-modified stage-specific embryonic antigen 4 (“SSEA4”) conjugated to diphtheria toxoid cross-reactive material 197 (“DT”) or an azido-modified SSEA4 analog conjugated to DT; and (2) α-galactosylceramide C34 or α-glucosylceramide C34.

Also provided is a method for treating cancer by administering the immunogenic composition to a patient. Examples of the cancer treatable by the method include breast, colon, pancreatic, prostate, bladder, and brain cancer.

Furthermore, a method is disclosed for treating a tumor by identifying a neoantigen expressed in the tumor, obtaining an immunogenic composition for raising an immune response against the neoantigen, and administering the immunogenic composition to the patient. The immunogenic composition includes a glycoconjugate containing (1) a fusion protein composed of an Fcγ fused to the neoantigen, the fusion protein being cross-linked to an azido-modified SSEA4 conjugated to DT or an azido-modified SSEA4 analog conjugated to DT; and (2) α-galactosylceramide C34 or α-glucosylceramide C34.

The details of several embodiments of the present invention are set forth in both the description and the drawing below. Other features, objects, and advantages of the invention will be apparent from the description and also from the appended claims. Finally, all references cited herein are hereby incorporated by reference in their entirety.

### BRIEF DESCRIPTION OF THE DRAWINGS

FIG. 1 shows the chemical structures of glycolipid adjuvants α-galactosylceramide (“αGalCer” or “C1”), derivatives of αGalCer, α-glucosylceramide (“αGlcCer”), and derivatives of αGlcCer.

### DETAILED DESCRIPTION

As mentioned, supra, the immunogenic composition of the invention includes a glycoconjugate. The glycoconjugate contains several components, among them being a fusion protein that includes an Fcγ fused to a TAA. The Fcγ can form the N-terminus of the fusion protein (“Fcγ-TAA”). Alternatively, the TAA can form the N-terminus of the fusion protein (“TAA-Fcγ”). Suitable methods for constructing and expressing Fcγ fusion proteins can be found in Lo et al. 1998, Protein Engineering 11:495-500.

In one aspect of the invention, the TAA is an antigen expressed highly in tumors and expressed at low levels or not at all in normal cells. Exemplary TAAs are lymphocyte antigen 6 complex, locus K (LY6K), cell division cycle associated 1 (CDCA1), insulin-like growth factor-II mRNA-binding protein 3 (IMP-3), kinesin family member 20A (KIF20A), glypican-3(GPC3), forkhead box M1 (FOXM1), cadherin 3 (CDH3), secreted protein acidic and rich in cysteine (SPARC), cell division cycle 45 ligand (CDC45L), DEP domain containing 1 (DEPDC1), M-phase phosphoprotein 1 (MPHOSPH1), prostate-specific antigen (PSA), prostate-specific membrane antigen (PSMA), human epidermal growth factor receptor2/neuroblastoma (HER2/neu), carcinoembryonic antigen (CEA), mutated epidermal growth factor receptor (EGFR), melanoma antigen (MAGE), mucin-1 (MUC-1), and New York esophageal squamous cell carcinoma 1 (NY-ESO-1). See Hirayama et al. 2016, Int. Immunol. Advance Access May 28 pp 1-26 (“Hirayama et al.”).

In another aspect of the invention, the TAA is a neoantigen. As mentioned above, a neoantigen arises from a somatic mutation present only in cancerous cells. The neoantigen can be identified in a patient's tumor tissue, e.g., by comparing gene expression patterns between normal tissue and tumor tissue. Exemplary methods for identifying neoantigens are set forth in Hirayama et al.

In one embodiment, the C-terminus of the Fcγ is fused in frame directly to the N-terminus of the TAA. In another embodiment, the Fcγ is fused to the TAA via a peptide linker. The peptide linker can be any peptide linker described in Chen et al. 2013, Adv. Drug Deliv. Rev. 65:1357-1369. Exemplary peptide linkers are GGGGS (SEQ ID NO: 1), GGGGSGGGGS (SEQ ID NO: 2), and GGGGSGGGGSGGGGS (SEQ ID NO: 3). Again, the fusion protein can have the orientation Fcγ-peptide linker-TAA or TAA-peptide linker-Fey.

In the immunogenic composition of the invention, either of the two fusion proteins described above, i.e., Fcγ-TAA and TAA-Fcγ, is cross-linked to an azido-modified SSEA4 conjugated to DT. SSEA4 can be conjugated to DT as described in U.S. Pat. No. 9,028,836.

It is known that modifying SSEA4 with an azido group improves the antigenicity of SSEA4. See Lee et al. The azido modification can be at the reducing end of SSEA4 or at its non-reducing end. Preferably, the azido modification is at the non-reducing end. Azido modification of SSEA4 can be carried out as also set forth in Lee et al.

SSEA4 analogs, e.g., SSEA3 and Globo H, can also be modified with an azido group at their reducing or non-reducing end again as described in Lee et al.

As mentioned above, the fusion proteins Fcγ-TAA and TAA-Fcγ can be cross-linked to the azido-modified SSEA4 conjugated to DT or to the azido-modified SSEA4 analog conjugated to DT.

For example, the Fcγ-TAA can be cross-linked via lysine residues to DT. In one embodiment, the Fey portion of the Fcγ-TAA is cross-linked to DT. Alternatively, the TAA portion of the Fcγ-TAA is cross-linked to DT. In other embodiments, either the TAA portion or the Fcγ portion of the TAA-Fcγ is cross-linked to DT.

Cross-linking between the fusion protein and DT can be carried out using a chemical cross-linking agent. Any chemical cross-linking agent described in Thermo Scientific Crosslinking Technical Handbook 2012 can be used to cross-link the fusion protein to DT. In certain embodiments, the chemical cross-linking agent is glutaraldehyde, dimethyl adipimidate, or dimethyl suberimidate.

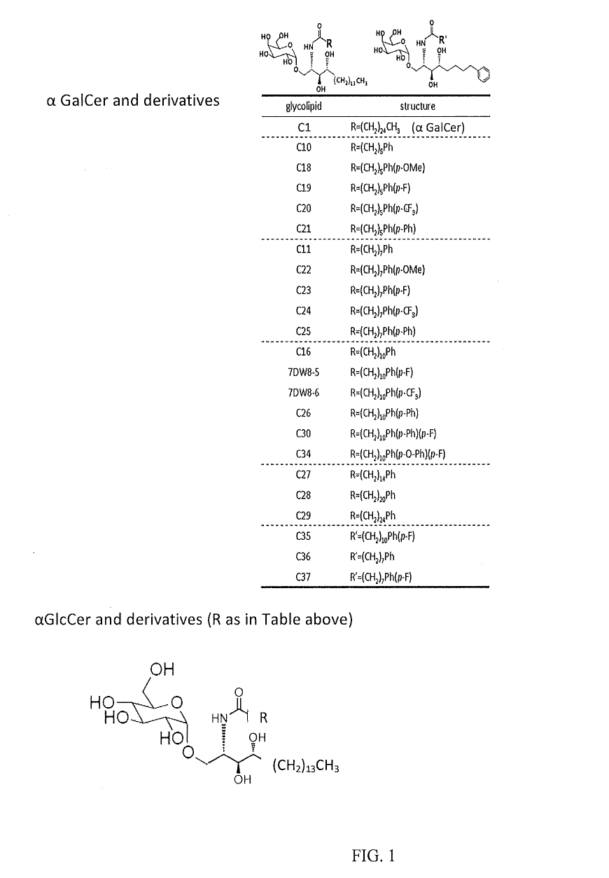

As set forth, supra, the immunogenic composition of the invention also includes α-galactosylceramide C34 or α-glucosylceramide C34. Alternatively, the immunogenic composition can include derivatives of α-galactosylceramide or derivatives of α-glucosylceramide. Exemplary derivatives of α-galactosylceramide and α-glucosylceramide are shown in FIG. 1 herein and described in International Application Publication 2008/128207 and in U.S. Pat. No. 9,028,836.

In a particular embodiment, the immunogenic composition of this invention includes (i) a glycoconjugate containing the fusion protein HER2/neu-Fcγ cross-linked via its HER2/neu portion to the DT part of azido-modified SSEA4 conjugated to DT, the azido modification being at the non-reducing end of SSEA4, and (ii) α-galactosylceramide C34.

Not to be bound by theory, it is believed that the Fcγ-TAA fusion protein portion of the glycoconjugate will localize the glycoconjugate to dendritic cells via Fc receptors on their surfaces. The TAA portion and the SSEA4 portion can be internalized by the dendritic cells and processed for antigen presentation, leading to antibody production. Specific antibodies against the TAA and against SSEA4 can directly bind to tumor cells to promote tumor eradication. Cytotoxic T lymphocytes (CTL) reactive against the TAA are also expected to be raised and to attack the tumor cells. The α-galactosylceramide C34 or α-glucosylceramide C34 acts as an adjuvant to further stimulate the immune reaction against the tumor that expresses the TAA and/or SSEA4.

Also disclosed is a method for treating cancer. The method includes administering to a subject in need thereof the immunogenic composition described above. Any of the embodiments of the immunogenic composition can be administered for treating cancer.

The cancer to be treated can be, but is not limited to breast, colon, gastrointestinal, kidney, lung, liver, ovarian, pancreatic, rectal, stomach, testicular, thymic, cervical, brain, prostate, bladder, skin, nasopharyngeal, esophageal, oral, head and neck, bone, cartilage, muscle, lymph node, and bone marrow cancer.

Further provided is a tumor treatment method that includes a step of identifying a neoantigen expressed in tumor cells of a subject. As mentioned above, the neoantigen can be identified by methods described in Hirayama et al. An immunogenic composition including the neoantigen is then provided. The immunogenic composition contains the neoantigen as well as the additional components set forth above, e.g., at page 3, first paragraph, supra. More specifically, the immunogenic composition includes both (i) a glycoconjugate containing a neoantigen-Fcγ fusion protein cross-linked via the neoantigen portion to the DT part of an azido-modified SSEA4-DT conjugate, the azido modification being at the non-reducing end of SSEA4; and (ii) α-galactosylceramide C34. The immunogenic composition containing the neoantigen is administered to the subject for treating the tumor in which the neoantigen was identified.

Without further elaboration, it is believed that one skilled in the art can, based on the description above, utilize the present invention to its fullest extent.

The following references, some cited supra, can be used to better understand the background of the application:
Berti et al. 2013, ACS Chemical Biol. 8:1653-1663
Buonaguro et al. 2011, Clin. Vaccine Imunol. 18:23-34
Chen et al. 2013, Adv. Drug Deliv. Rev. 65:1357-1369
Cheung et al. 2016, Proc. Natl. Acad. Sci. 113:960-965
Chiang et al. 2015, Vaccines 3:344-372
Danishefsky et al. 2015, Acc. Chem. Res. 48:643-652
DiLillo et al. 2015, Cancer Immunol. Res. 3:704-713
Hirayama et al. 2016, Int. Immunol. Advance Access May 28 pp 1-26
Huang et al. 2013, Proc. Natl. Acad. Sci. 110:2517-2522
Lee et al. 2014, J. Amer. Chem. Soc. 136:16844-16853
Liu et al. 2008, Eur. J. Immunol. 38:1012-1023
Lo et al. 1998, Protein Engineering 11:495-500.
Melero et al. 2014, Nature Rev. Oncol. 11:509-524
Thermo Scientific Crosslinking Technical Handbook 2012

The contents of the above references are hereby incorporated by reference in their entirety.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

1. An immunogenic composition for treating cancer, comprising:
a glycoconjugate containing a fusion protein that includes an immunoglobulin gamma Fc domain (Fcγ) fused to a tumor-associated antigen (TAA), the fusion protein being cross-linked to an azido-modified stage-specific embryonic antigen 4 (SSEA4) conjugated to diphtheria toxoid cross-reactive material 197 (DT) or an azido-modified SSEA4 analog conjugated to DT; and
α-galactosylceramide C34 or α-glucosylceramide C34,
wherein the immunogenic composition stimulates a T-cell response to the TAA, to SSEA4 or the SSEA4 analog, or to both the TAA and SSEA4 or the SSEA4 analog.

2. The immunogenic composition of claim 1, wherein the TAA is lymphocyte antigen 6 complex, locus K (LY6K), cell division cycle associated 1 (CDCA1), insulin-like growth factor-II mRNA-binding protein 3 (IMP-3), kinesin family member 20A (KIF20A), glypican-3(GPC3), forkhead box M1 (FOXM1), cadherin 3 (CDH3), secreted protein acidic and rich in cysteine (SPARC), cell division cycle 45 ligand (CDC45L), DEP domain containing 1 (DEPDC1), M-phase phosphoprotein 1 (MPHOSPH1), prostate-specific antigen (PSA), prostate-specific membrane antigen (PSMA), human epidermal growth factor receptor 2/neuroblastoma (HER2/neu), carcinoembryonic antigen (CEA), mutated epidermal growth factor receptor (EGFR), melanoma antigen (MAGE), mucin-1 (MUC-1), or New York esophageal squamous cell carcinoma 1 (NY-ESO-1).

3. The immunogenic composition of claim 2, wherein the Fcγ forms the N-terminus of the fusion protein.

4. The immunogenic composition of claim 3, wherein the TAA is cross-linked to the DT.

5. The immunogenic composition of claim 4, wherein the azido modification is at the non-reducing end of the SSEA4 or the SSEA4 analog.

6. The immunogenic composition of claim 2, wherein the TAA forms the N-terminus of the fusion protein.

7. The immunogenic composition of claim 6, wherein the TAA is cross-linked to the DT.

8. The immunogenic composition of claim 7, wherein the azido modification is at the non-reducing end of the SSEA4 or the SSEA4 analog.

9. The immunogenic composition of claim 1, wherein the TAA is a neoantigen.

10. The immunogenic composition of claim 9, wherein the Fcγ forms the N-terminus of the fusion protein.

11. The immunogenic composition of claim 10, wherein the TAA is cross-linked to the DT.

12. The immunogenic composition of claim 11, wherein the azido modification is at the non-reducing end of the SSEA4 or the SSEA4 analog.

13. The immunogenic composition of claim 9, wherein the TAA forms the N-terminus of the fusion protein.

14. The immunogenic composition of claim 13, wherein the Fcγ is cross-linked to the DT.

15. The immunogenic composition of claim 14, wherein the azido modification is at the non-reducing end of the SSEA4 or the SSEA4 analog.

16. A method for treating cancer, the method comprising administering to a subject having a malignant tumor the immunogenic composition of claim 1, wherein the tumor expresses the tumor-associated antigen.

17. The method of claim 16, wherein the cancer is breast, colon, gastrointestinal, kidney, lung, liver, ovarian, pancreatic, rectal, stomach, testicular, thymic, cervical, prostate, bladder, skin, nasopharyngeal, esophageal, oral, head and neck, bone, cartilage, muscle, lymph node, bone marrow, or brain cancer.

18. A method for treating cancer, the method comprising administering to a subject having a malignant tumor the immunogenic composition of claim 8, wherein the TAA is HER2/neu and the cancer is breast cancer.

19. A method for treating a tumor in a subject, the method comprising:
identifying a neoantigen in the tumor of the subject,
obtaining an immunogenic composition that includes:
a glycoconjugate containing a fusion protein that includes an immunoglobulin Fc domain fused to the neoantigen, the fusion protein being attached via a linker to an azido-modified SSEA4 conjugated to diphtheria toxoid cross-reactive material 197 or an azido-modified SSEA4 analog conjugated to diphtheria toxoid cross-reactive material 197; and
α-galactosylceramide C34 or α-glucosylceramide C34, and
administering the immunogenic composition to the subject, thereby stimulating a T-cell response against the tumor.

20. The method of claim 19, wherein the tumor is a breast, colon, gastrointestinal, kidney, lung, liver, ovarian, pancreatic, rectal, stomach, testicular, thymic, cervical, prostate, bladder, skin, nasopharyngeal, esophageal, oral, head and neck, bone, cartilage, muscle, lymph node, bone marrow, or brain tumor.
